(19) United States
(12) Patent Application Publication
(10) Pub. No.: US 2023/0329905 A1
(43) Pub. Date: Oct. 19, 2023

(54) Eye Drop Dispenser

(71) Applicant: DoseLogix, LLC, Woodstock, GA (US)

(72) Inventors: David C. Gale, Kennesaw, GA (US); Stephen D. M. Jones, Doraville, GA (US); Kevin B. Green, Smyrna, GA (US); Matthew Beyer, Acworth, GA (US); Maureen B. Carroll, Atlanta, GA (US); Craig Cochran, Atlanta, GA (US)

(73) Assignee: DoseLogix, LLC, Woodstock, GA (US)

(21) Appl. No.: 18/134,543

(22) Filed: Apr. 13, 2023

Related U.S. Application Data
(60) Provisional application No. 63/330,946, filed on Apr. 14, 2022.

(51) Int. Cl. A61F 9/00

(52) U.S. Cl. CPC A61F 9/0026

(57) ABSTRACT

An eye drop dispenser having a dispensing body configured to be removably secured to a bottle holding a dispensing solution. The dispenser includes a dispensing tip in fluid communication with the bottle for dispensing solution that flows from the bottle out the dispensing tip. The dispenser includes a cheek rest arm that includes a curved portion configured to be positioned adjacent a cheekbone of a user and a cheek rest portion that is positioned along a length of a cheek of the user when the curved portion of the cheek rest arm is positioned adjacent the cheekbone of the user. When the dispensing body is secured to the bottle and the curved portion of the cheek rest arm is positioned adjacent the cheekbone of the user, the dispensing tip is positioned adjacent an eye of the user for dispensing solution into the eye by gravity.

### CROSS REFERENCE TO RELATED APPLICATION

This application claims priority to co-pending U.S. Provisional Application Ser. No. 63/330,946 filed Apr. 14, 2022, entitled “Eye Drop Dispenser,” the entire contents of which is incorporated herein by reference.

### FIELD

The present disclosure is directed to an eye drop dispenser. More specifically, the present disclosure is directed to an eye drop dispenser having a cheek rest arm.

### BACKGROUND

Devices for dispensing eye drops are known in the art. Problems with existing devices include difficulty in properly positioning the device with respect to a user's eye and difficulty in squeezing or otherwise operating the device to dispense the appropriate amount of liquid in the user's eye once in the desired position. As a result, a user will often waste a significant portion of the medication in the eye drop dispenser because the appropriate amount of solution either missed the user's eye or was wasted due to the tendency to over-squeeze the squeeze bottle. These difficulties are particularly problematic for more senior users of the devices or users that otherwise have shaky/unstable hands.

What is needed therefore is a more ergonomic eye drop dispenser that provides for consistent and accurate positioning of the dispenser with respect to an eye of the user as well as accurate dosing of the liquid medication being dispensed.

### SUMMARY

The above and other needs are met by an eye drop dispenser including a bottle having an internal chamber for holding a solution and a conduit having a first end and a second end. The second end of the conduit is operable to be inserted into the internal chamber of the bottle. A dispensing body having a flow channel extends through the dispensing body from a proximal end to a distal end, and the dispensing body is configured to be removably secured to the bottle with the first end of the conduit fluidly connected to the proximal end of the flow channel and the second end of the conduit inserted into the internal chamber of the bottle. A dispensing tip is in fluid communication with the distal end of the flow channel of the dispensing body for dispensing solution that flows from the internal chamber of the bottle through the flow channel and out the dispensing tip. A cheek rest arm extends from the dispensing body with the cheek rest arm having a curved portion configured to be positioned adjacent a cheekbone of a user. When the dispensing body is secured to the bottle and the curved portion of the cheek rest arm is positioned adjacent the cheekbone of the user, the dispensing tip is positioned adjacent an eye of the user for dispensing solution into the eye by gravity.

According to certain embodiments, the flow channel and dispensing tip are dimensioned and configured to dispense the solution in a direction substantially perpendicular to a longitudinal axis of the bottle.

According to certain embodiments, the cheek rest arm further includes a cheek rest portion that is positioned along a length of a cheek of the user when the curved portion of the cheek rest arm is positioned adjacent the cheekbone of the user. In some embodiments, the curved portion of the cheek rest arm is contoured to substantially fit against a bottom surface of the cheekbone adjacent a nose of the user when the curved portion of the cheek rest arm is positioned adjacent the cheekbone of the user. According to some embodiments, the cheek rest arm is dimensioned and configured such that a positioning of a distal end of the cheek rest portion of the cheek rest arm corresponds to a bottom surface of the bottle when the bottom surface of the bottle is positioned on a flat surface.

According to certain embodiments, the cheek rest arm is dimensioned and configured to operate as a stand for the eye drop dispenser when a bottom surface of the bottle is positioned on a flat surface.

According to certain embodiments, the cheek rest arm is integrally formed as part of the dispensing body.

According to another embodiment of the disclosure, an eye drop dispenser includes a dispensing body having a dispensing tip and a cheek rest arm. The cheek rest arm includes a curved portion configured to be positioned adjacent a cheekbone of a user and a cheek rest portion that is positioned along a length of a cheek of the user when the curved portion of the cheek rest arm is positioned adjacent the cheekbone of the user. When the dispensing body is secured to a bottle and the curved portion of the cheek rest arm is positioned adjacent the cheekbone of the user, the dispensing tip is positioned adjacent an eye of the user for dispensing solution into the eye by gravity.

According to certain embodiments, when the dispensing body is secured to the bottle, the dispensing body and dispensing tip are dimensioned and configured to dispense a solution from the bottle in a direction substantially perpendicular to a longitudinal axis of the bottle.

According to certain embodiments, the cheek rest arm includes a connecting portion that extends out with respect to the dispensing body and the curved portion curves downward from the connecting portion. In some embodiments, the curved portion of the cheek rest arm is contoured to substantially fit against a bottom surface of the cheekbone adjacent a nose of the user when the curved portion of the cheek rest arm is positioned adjacent the cheekbone of the user.

According to certain embodiments, the cheek rest arm is dimensioned and configured such that a positioning of a distal end of the cheek rest portion of the cheek rest arm corresponds to a bottom surface of the bottle when the dispensing body is secured to the bottle and the bottom surface of the bottle is positioned on a flat surface.

According to certain embodiments, the cheek rest arm is dimensioned and configured to operate as a stand for the eye drop dispenser when the dispensing body is secured to the bottle and a bottom surface of the bottle is positioned on a flat surface.

According to certain embodiments, the cheek rest arm is integrally formed as part of the dispensing body.

According to another embodiment of the disclosure, an eye drop dispenser includes a squeeze bottle having an internal chamber for holding a solution. A conduit having a first end and a second end is operable to be inserted into the internal chamber of the squeeze bottle. A dispensing body having a flow channel extending through the dispensing body from a proximal end to a distal end is configured to be removably secured to a neck of the squeeze bottle with the first end of the conduit fluidly connected to the proximal end of the flow channel and the second end of the conduit inserted into the internal chamber of the squeeze bottle. A dispensing tip is in fluid communication with the distal end of the flow channel of the dispensing body for dispensing solution that flows from the internal chamber of the squeeze bottle through the flow channel and out the dispensing tip, the flow channel and dispensing tip being dimensioned and configured to dispense the solution in a direction substantially perpendicular to a longitudinal axis of the bottle. A cheek rest arm having a curved portion is configured to be positioned adjacent a cheekbone of a user. The cheek rest arm further includes a cheek rest portion that is positioned along a length of a cheek of the user when the curved portion of the cheek rest arm is positioned adjacent the cheekbone of the user. When the dispensing body is secured to the neck of the bottle and the curved portion of the cheek rest arm is positioned adjacent the cheekbone of the user, the dispensing tip is positioned adjacent an eye of the user for dispensing solution into the eye by gravity.

According to certain embodiments, the cheek rest arm is dimensioned and configured such that a positioning of a distal end of the cheek rest portion of the cheek rest arm corresponds to a bottom surface of the squeeze bottle when the bottom surface of the bottle is positioned on a flat surface.

According to certain embodiments, the cheek rest arm is dimensioned and configured to operate as a stand for the eye drop dispenser when the dispensing body is secured to the squeeze bottle and a bottom surface of the bottle is positioned on a flat surface.

According to certain embodiments, the cheek rest arm is integrally formed as part of the dispensing body.

According to certain embodiments, the curved portion of the cheek rest arm is contoured to substantially fit against a bottom surface of the cheekbone adjacent a nose of the user when the curved portion of the cheek rest arm is positioned adjacent the cheekbone of the user.

### BRIEF DESCRIPTION OF THE DRAWINGS

Other embodiments of the disclosure will become apparent by reference to the Detailed Description in conjunction with the figures, wherein elements are not necessarily to scale so as to more clearly show the details, wherein like reference numbers indicate like elements throughout the several views, and wherein:

FIG. 1 depicts a front perspective view of an eye drop dispenser according to one embodiment of the disclosure.

FIG. 2 depicts a side elevational view of the component parts of an eye drop dispenser according to one embodiment of the disclosure.

FIG. 3 depicts a side cross-sectional view of an eye drop dispenser according to one embodiment of the disclosure.

FIG. 4 depicts a side view of an eye drop dispenser according to one embodiment of the disclosure in position for dispensing eye drops with respect to an eye of a user.

### DETAILED DESCRIPTION

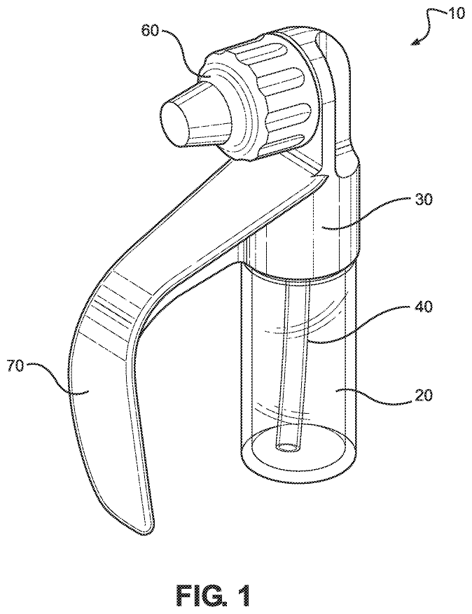

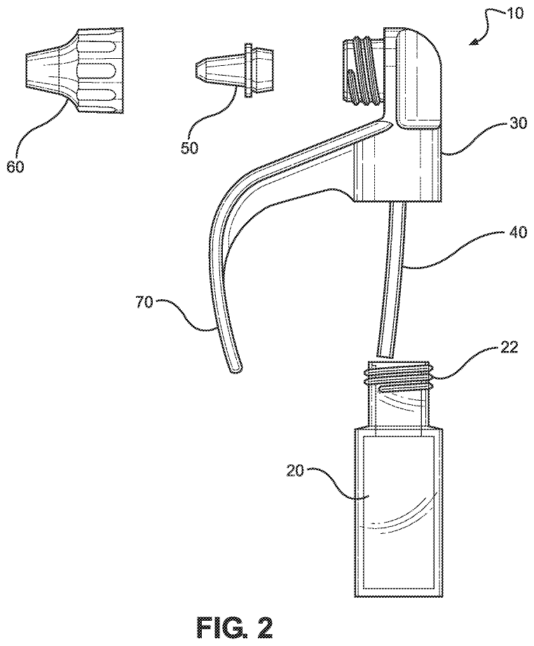

With reference to FIGS. 1-2, an eye drop dispenser 10 according to one embodiment of the disclosure is depicted. The dispenser 10 generally includes a bottle 20 having an internal chamber for holding an eye solution, a dispensing body 30 that is configured to be removably attached to a neck 22 of bottle 20 (e.g., using a threaded, press-fit or other type of engagement as known in the art), a dip tube 40, a dispensing tip 50, and an optional cap 60 for covering the dispensing tip 50 when the dispenser 10 is not being used. In certain embodiments, the dispensing tip 50 may be removably secured to the dispensing body 30 as shown herein and described below. In other embodiments, the dispensing tip 50 may be formed as an integral part of the dispensing body 30. The bottle 20 is preferably a squeeze bottle such that the eye drop dispenser 10 is configured to dispense eye drops from the dispensing tip 50 based on a squeezing action by the user upon the bottle 20. However, other types of bottles and accompanying dispensing actions of the dispenser 10 are contemplated and intended to be within the scope of the disclosure (e.g., dispensers using capillary, trigger, plunger, etc. type actions).

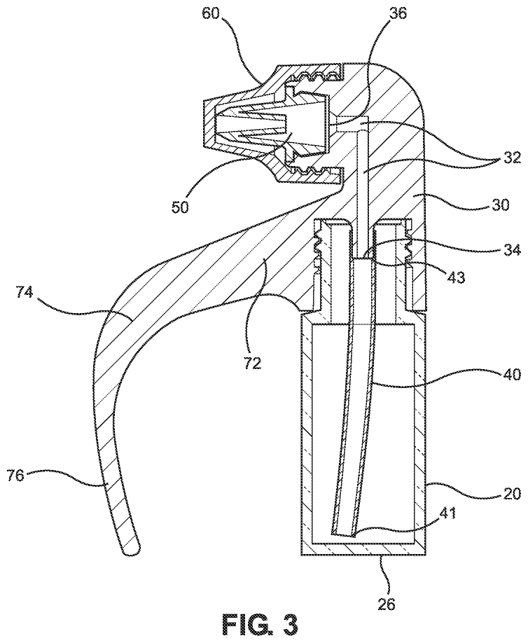

With reference to the cross-sectional view of FIG. 3, the dispensing body 30 includes a flow channel 32 that extends through the interior of the dispensing body 30 from a proximal end 34 to a distal end 36. The dip tube 40 is fluidly connected at a first end 43 to the proximal end 34 of flow channel 32. The dispensing tip 50 is then fluidly connected to or otherwise in fluid communication with the distal end 36 of the flow channel 32. In operation, the squeeze bottle 20 houses the eye solution to be dispensed. The second end 41 of the dip tube 40 is then inserted into the squeeze bottle 20 such that the interior of the squeeze bottle 20 is in fluid communication with the dispensing tip 50 via the flow channel 32. With the dip tube 40 properly inserted into the squeeze bottle 20, the dispensing body 30 is then secured to the squeeze bottle 20. When the dispenser 10 is positioned such that the dispensing tip 50 is held over an eye of the user and the squeeze bottle 20 is squeezed, an appropriate amount of eye solution travels from the dip tube 40 through the flow channel 32 and out the dispensing tip 50.

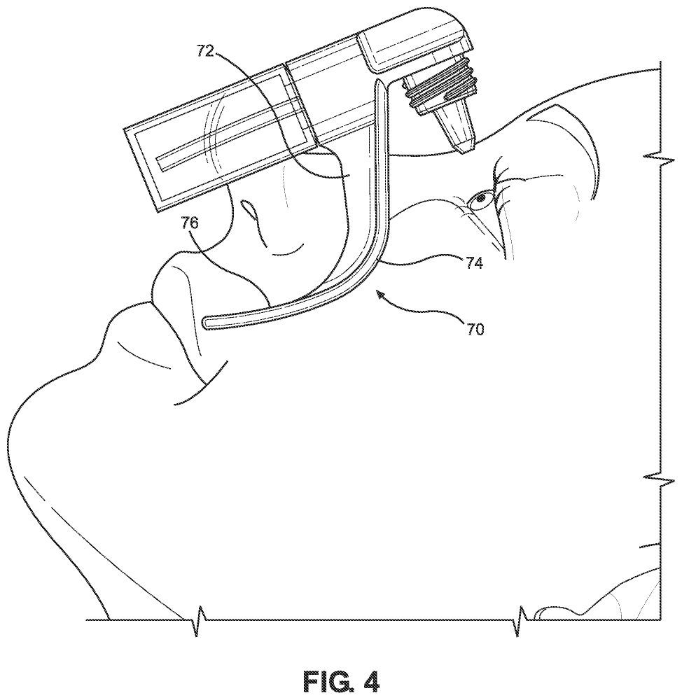

As shown, the dispensing body 30 and corresponding dispensing tip 50 are preferably configured such that the eye solution is dispensed from the dispensing tip 50 in a direction that is substantially perpendicular to the longitudinal axis of the squeeze bottle 20. In other words, instead of being released vertically from the eye drop dispenser 10, the eye drop dispenser 10 is configured to dispense the eye solution in a horizontal direction as compared to the longitudinal axis of the squeeze bottle 20. As a result of the perpendicular relationship between the dispensing tip 50 and the squeeze bottle 20, the dispenser 10 of the present disclosure is dimensioned and configured for the dispensing tip 10 to be positioned directly above an eye of a user with a relatively modest head tilt (see FIG. 4). The dispensing solution then travels from the dispensing tip 50 into the eye via gravity.

In preferred embodiments, the eye drop dispenser 10 further includes a cheek rest arm 70 generally positioned underneath the dispensing tip 50. The cheek rest arm 70 is dimensioned and configured to extend out from the squeeze bottle generally in the same horizontal direction as the dispensing tip 50. With reference to FIG. 3, the cheek rest arm 70 preferably includes at least a curved portion 74 and a cheek rest portion 76. In operation, and with reference to FIG. 4, the cheek rest arm 70 is dimensioned and configured such that, when the curved portion 74 is positioned adjacent the cheekbone (also called the zygomatic or malar bone) of a user, the dispensing tip 50 is appropriately positioned above the eye of the user upon a modest head tilt to receive the dispensing solution and the cheek rest portion 76 is positioned along the length of the user's cheek to help stabilize the dispensing tip 50.

With reference back to FIG. 3, the cheek rest arm 70 further includes a connection portion 72 that connects the curved portion 74 and cheek rest portion 76 of the cheek rest arm 70 to the dispensing body 30. As shown, the connection portion 72 of this embodiment is positioned below the dispensing tip 50 and generally extends out from the dispensing body 30 in the same direction as the dispensing tip 50. More specifically, in this embodiment, the connection portion 72 extends at a slight downward angle with respect to the dispensing tip 50. The curved portion 74 then curves downward from the connection portion 72 such that the curvature of the curved portion 74 is generally contoured to be pressed up against the cheekbone of a user when the curved portion 74 is positioned next to the nose of the user. As shown, the connection portion 72 preferably extends from the squeeze bottle 20 further than the dispensing tip 50. Accordingly, when the curved portion 74 is positioned against the cheekbone of a user, the dispensing tip 50 is appropriately spaced from the eye of the user as shown in FIG. 4.

According to another aspect of the disclosure, the cheek rest portion 76 of the cheek rest preferably extends downward from the curved portion 74 at a length that corresponds to the bottom 26 of the squeeze bottle 20 when the dispenser 10 is in an upright position (i.e., where the bottom 26 of the bottle 20 is positioned on a flat surface). As a result, cheek rest arm 70 may be used as a stand to help prevent the dispenser 10 from tipping over when not in use.

It should be understood that the dispensing body 30 and corresponding connection portion 72 for the cheek rest arm 70 could take many different forms within the scope of the present disclosure. For example, the dispensing body 30 and connection portion 70 could be dimensioned and configured such that the connection portion 70 extends out from the bottom of the bottle 20 with the cheek rest arm 70 then extending upward towards the dispensing tip 50 with the cheek rest portion 76 disposed between the connection portion 70 and curved portion 74. According to this embodiment, the connection portion 70 would preferably be configured to provide the stand function of the dispenser 10.

As shown herein, the cheek rest arm 70 may be integrally formed as part of the dispensing body 30. Alternatively, it may be a separate component that is removably secured to the dispenser 10 such that it can be used on an as needed/desired basis. For example, the cheek rest arm 70 could include an attachment ring that is intended to fit around the neck of the squeeze bottle 20. According to this embodiment, the cheek rest arm 70 is secured to the dispenser 10 by first fitting the ring around the neck of the squeeze bottle 20 and then securing the dispensing body 30 to the neck of the squeeze bottle 20 such that the cheek rest arm 70 is held in place by the dispensing body 30. Alternately, the cheek rest arm 70 could be removably secured directly to the dispensing body 30 such as with a threaded or press fit engagement.

In certain embodiments, the dispensing body 30 with integrated cheek rest arm 70 is provided as a component of an overall eye drop dispenser 10 that includes the bottle 20. However, in other embodiments, the dispensing body 30 (with associated dip tube 40) may be provided as an independent component used to modify an existing bottle 20.

With reference to FIG. 4, usage of the dispenser 10 with cheek rest arm 70 is further described as follows: (1) the user performs achieves a modest head-tilt angle (the necessary head-tilt being more easily achievable by a wider range of users using the dispenser 10 of the present disclosure); (2) upon tilting, the cheek rest arm 70 is positioned on the cheek of the user to achieve and maintain proper positioning of the dispensing tip 50 above the eye of the user (alternately, the cheek rest arm 70 may be lightly pressed against the cheek of the user before the user tilts their head backwards); (3) the user can pivot the dispenser 10 using the curved portion 74 of the cheek rest arm 70 to further adjust the positioning of the dispensing tip 50 with respect to the eye of the user; and (4) with the cheek rest arm 70 resting against the cheek of the user, the user is able to squeeze the squeeze bottle 20 from a more relaxed hand position to dispense the appropriate amount of dispensing solution (e.g., number of drops) into the eye of the user by gravity. For purposes of the present disclosure, “by gravity” refers to the gravitational force acting on the dispensing solution (e.g., eye drops) once the solution is dispensed from the dispensing tip 50.

The shape of the cheek rest arm 70 incorporated into the dispenser 10 as shown and described herein provides for several ergonomic benefits. As noted above, the cheek rest arm 70 provides for a more relaxed/modest head tilt. Further, the cheek rest arm 70 provides for precise and stabilized positioning of the dispensing tip 50 with respect to the eye of the user. The cheek rest arm 70 also allows for steady control for users with shaky hands and promotes a consistent and normal squeeze reflex. In this regard, the cheek rest arm 70 promotes less arm strain/stress due to the lower position of the squeeze bottle 20 (i.e., squeeze bottle 20 is generally parallel to the user's cheek instead of having to be held above the user's eye). Additionally, the cheek rest arm 70 minimizes unwanted movement of the dispensing tip 50 due to a flinch reflex. All of the above promotes consistent and accurate dosing by providing and maintaining proper positioning of the dispensing tip 50 during dispensing and preventing a tendency for over-squeezing of the squeeze bottle 20. This in turn provides cost savings due to less wasted medication. It also helps to reduce the potential for contamination by helping to prevent the dispensing tip 50 from contacting the eye.

The foregoing description of preferred embodiments for this disclosure have been presented for purposes of illustration and description. They are not intended to be exhaustive or to limit the disclosure to the precise form disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the disclosure in various embodiments and with various modifications as are suited to the particular use contemplated. All such modifications and variations are within the scope of the disclosure as determined by any future claims when interpreted in accordance with the breadth to which they are fairly, legally, and equitably entitled.

What is claimed is:

1. An eye drop dispenser including:
a bottle having an internal chamber for holding a solution;
a conduit having a first end and a second end, the second end for inserting into the internal chamber of the bottle;
a dispensing body having a flow channel extending through the dispensing body from a proximal end to a distal end, the dispensing body configured to be removably secured to the bottle with the first end of the conduit fluidly connected to the proximal end of the flow channel and the second end of the conduit inserted into the internal chamber of the bottle;
a dispensing tip in fluid communication with the distal end of the flow channel of the dispensing body for dispensing solution that flows from the internal chamber of the bottle through the flow channel and out the dispensing tip; and
a cheek rest arm extending from the dispensing body, the cheek rest arm having a curved portion configured to be positioned adjacent a cheekbone of a user,
wherein, when the dispensing body is secured to the bottle and the curved portion of the cheek rest arm is positioned adjacent the cheekbone of the user, the dispensing tip is positioned adjacent an eye of the user for dispensing solution into the eye by gravity.

2. The eye drop dispenser of claim 1 wherein the flow channel and dispensing tip are dimensioned and configured to dispense the solution in a direction substantially perpendicular to a longitudinal axis of the bottle.

3. The eye drop dispenser of claim 1 wherein the cheek rest arm further includes a cheek rest portion that is positioned along a length of a cheek of the user when the curved portion of the cheek rest arm is positioned adjacent the cheekbone of the user.

4. The eye drop dispenser of claim 3 wherein the curved portion of the cheek rest arm is contoured to substantially fit against a bottom surface of the cheekbone adjacent a nose of the user when the curved portion of the cheek rest arm is positioned adjacent the cheekbone of the user.

5. The eye drop dispenser of claim 4 wherein the cheek rest arm is dimensioned and configured such that a positioning of a distal end of the cheek rest portion of the cheek rest arm corresponds to a bottom surface of the bottle when the bottom surface of the bottle is positioned on a flat surface.

6. The eye drop dispenser of claim 1 wherein the cheek rest arm is dimensioned and configured to operate as a stand for the eye drop dispenser when a bottom surface of the bottle is positioned on a flat surface.

7. The eye drop dispenser of claim 1 wherein the cheek rest arm is integrally formed as part of the dispensing body.

8. An eye drop dispenser comprising:
a dispensing body having a dispensing tip; and
a cheek rest arm having a curved portion configured to be positioned adjacent a cheekbone of a user and a cheek rest portion that is positioned along a length of a cheek of the user when the curved portion of the cheek rest arm is positioned adjacent the cheekbone of the user, and
wherein, when the dispensing body is secured to a bottle and the curved portion of the cheek rest arm is positioned adjacent the cheekbone of the user, the dispensing tip is positioned adjacent an eye of the user for dispensing solution into the eye by gravity.

9. The eye drop dispenser of claim 8 wherein, when the dispensing body is secured to the bottle, the dispensing body and dispensing tip are dimensioned and configured to dispense a solution from the bottle in a direction substantially perpendicular to a longitudinal axis of the bottle.

10. The eye drop dispenser of claim 8 wherein the cheek rest arm includes a connecting portion that extends out with respect to the dispensing body and the curved portion curves downward from the connecting portion.

11. The eye drop dispenser of claim 10 wherein the curved portion of the cheek rest arm is contoured to substantially fit against a bottom surface of the cheekbone adjacent a nose of the user when the curved portion of the cheek rest arm is positioned adjacent the cheekbone of the user.

12. The eye drop dispenser of claim 8 wherein the cheek rest arm is dimensioned and configured such that a positioning of a distal end of the cheek rest portion of the cheek rest arm corresponds to a bottom surface of the bottle when the dispensing body is secured to the bottle and the bottom surface of the bottle is positioned on a flat surface.

13. The eye drop dispenser of claim 8 wherein the cheek rest arm is dimensioned and configured to operate as a stand for the eye drop dispenser when the dispensing body is secured to the bottle and a bottom surface of the bottle is positioned on a flat surface.

14. The eye drop dispenser of claim 8 wherein the cheek rest arm is integrally formed as part of the dispensing body.

15. An eye drop dispenser including:
a squeeze bottle having an internal chamber for holding a solution;
a conduit having a first end and a second end, the second end for inserting into the internal chamber of the squeeze bottle;
a dispensing body having a flow channel extending through the dispensing body from a proximal end to a distal end, the dispensing body configured to be removably secured to a neck of the squeeze bottle with the first end of the conduit fluidly connected to the proximal end of the flow channel and the second end of the conduit inserted into the internal chamber of the squeeze bottle;
a dispensing tip in fluid communication with the distal end of the flow channel of the dispensing body for dispensing solution that flows from the internal chamber of the squeeze bottle through the flow channel and out the dispensing tip, the flow channel and dispensing tip being dimensioned and configured to dispense the solution in a direction substantially perpendicular to a longitudinal axis of the squeeze bottle; and
a cheek rest arm having a curved portion configured to be positioned adjacent a cheekbone of a user and a cheek rest portion that is configured to be positioned along a length of a cheek of the user when the curved portion of the cheek rest arm is positioned adjacent the cheekbone of the user,
wherein, when the dispensing body is secured to the neck of the squeeze bottle and the curved portion of the cheek rest arm is positioned adjacent the cheekbone of the user, the dispensing tip is positioned adjacent an eye of the user for dispensing solution into the eye by gravity.

16. The eye drop dispenser of claim 15 wherein the cheek rest arm is dimensioned and configured such that a positioning of a distal end of the cheek rest portion of the cheek rest arm corresponds to a bottom surface of the squeeze bottle when the bottom surface of the bottle is positioned on a flat surface.

17. The eye drop dispenser of claim 15 wherein the cheek rest arm is dimensioned and configured to operate as a stand for the eye drop dispenser when the dispensing body is secured to the bottle and a bottom surface of the squeeze bottle is positioned on a flat surface.

18. The eye drop dispenser of claim 15 wherein the cheek rest arm is integrally formed as part of the dispensing body.

19. The eye drop dispenser of claim 15 wherein the curved portion of the cheek rest arm is contoured to substantially fit against a bottom surface of the cheekbone adjacent a nose of the user when the curved portion of the cheek rest arm is positioned adjacent the cheekbone of the user.
